(19) United States
(12) Patent Application Publication
(10) Pub. No.: US 2019/0039999 A1
(43) Pub. Date: Feb. 7, 2019

(54) CELL SEEDING PLATE, METHOD FOR MANUFACTURING THE SAME, AND CELL SHEET SEPARATING METHOD

(71) Applicant: HON HAI PRECISION INDUSTRY CO., LTD., New Taipei (TW)

(72) Inventor: HSIU-WEN CHIEN, New Taipei (TW)

(21) Appl. No.: 15/689,002

(22) Filed: Aug. 29, 2017

(30) Foreign Application Priority Data
Aug. 4, 2017 (TW) 106126485
Aug. 25, 2017 (CN) 201710742467.7

(51) Int. Cl. C07C 233/11; C07C 205/04; C07C 69/145; C07C 43/164

(52) U.S. Cl. CPC C07C 233/11; C07C 205/04; C08F 16/36; C07C 43/164; C07C 69/145

(57) ABSTRACT

A cell seeding plate comprises a substrate and a photolysis layer formed on a surface of the substrate. The photolysis layer comprises a plurality of photolysis groups, and each of the plurality of photolysis groups has a chemical structural formula of

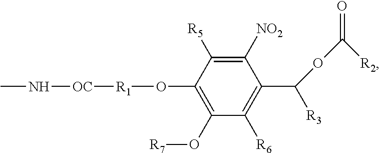

R₁ and R₃ each represents alkane group, R₂ comprises alkane group or olefin group, R₅, R₆ and R₇ each represents hydrogen group or alkane group. Each of the plurality of photolysis groups is bonded to the surface by the amide group.

### FIELD

The subject matter herein generally relates to a cell seeding plate, a method for manufacturing the cell seeding plate, and a cell sheet separating method using the cell seeding plate.

### BACKGROUND

Cells are usually seeded on a thermoresponsive plate to form a cell sheet. The whole thermoresponsive plate can be deformed when cooled, thus allowing the cell sheet to be separated from the thermoresponsive plate. However, an activity of the cells may be decreased when cooled.

### BRIEF DESCRIPTION OF THE DRAWINGS

Implementations of the present technology will now be described, by way of example only, with reference to the attached figures.

FIG. 1 is a diagram of an exemplary embodiment of a cell seeding plate.

FIG. 2 is a flowchart of an exemplary embodiment of a method for manufacturing a cell seeding plate.

FIG. 3 is a flowchart of an exemplary embodiment of a cell sheet separating method using the cell seeding plate.

FIG. 4 is a diagram of an exemplary embodiment of a cell layer formed on the cell seeding plate.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure.

The term “substantially” is defined to be essentially conforming to the particular dimension, shape or other word that substantially modifies, such that the component need not be exact. For example, substantially cylindrical means that the object resembles a cylinder, but can have one or more deviations from a true cylinder. The term “comprising,” when utilized, means “including, but not necessarily limited to”; it specifically indicates open-ended inclusion or membership in the so-described combination, group, series, and the like.

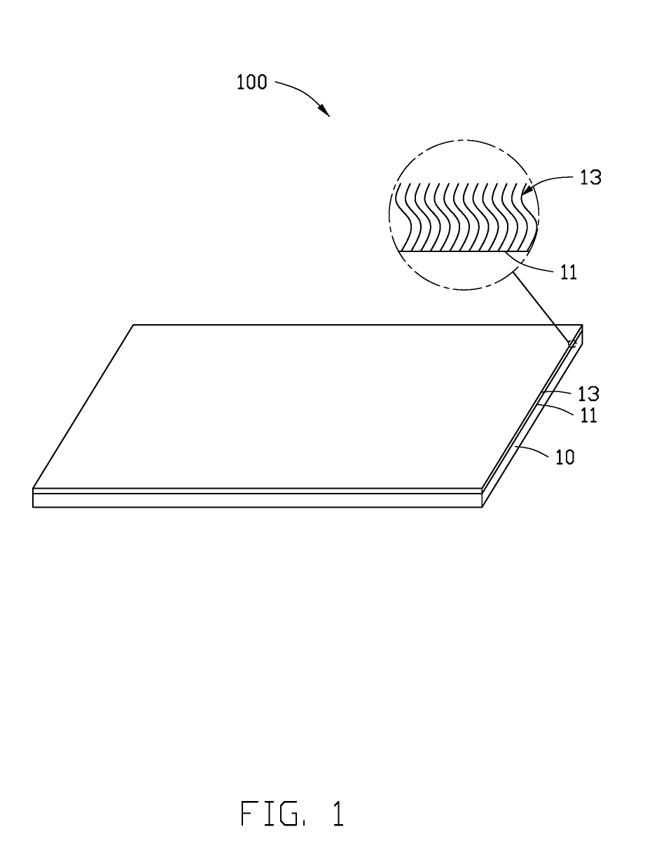

FIG. 1 illustrates an exemplary embodiment of a cell seeding plate 100. The cell seeding plate 100 comprises a substrate 10 and a photolysis layer 13 formed on a surface 11 of the substrate 10.

The substrate 10 is transparent, and made of plastic or glass, such as polystyrene.

The photolysis layer 13 comprises a plurality of photolysis groups. Each photolysis group has a chemical structural formula of

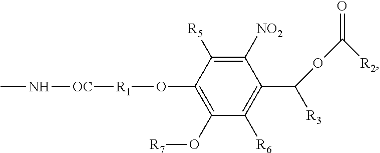

wherein R₁ and R₃ each represents alkane group, R₂ comprises alkane group or olefin group, R₅, R₆ and R₇ each represents hydrogen group or alkane group. Each photolysis group is bonded to the surface 11 by an amide group of the photolysis group. Ester group located at the ortho-position of nitro group of the photolysis group can be broken when under ultraviolet illumination, which can be shown as follows:

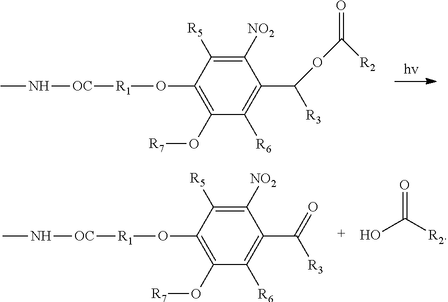

In at least one exemplary embodiment, R₂ may further comprise sulfur group. For example, R₂ may be a natural polymer comprising sulfur group, or a synthetic polymer comprising sulfur group. The natural polymer may be collagen, hyaluronic acid, protein for functionalizing cell, or peptides for functionalizing cell.

In at least one exemplary embodiment, R₁ is propyl group, R₂ is vinyl group, R₃ is methyl group, R₅ and R₆ are hydrogen group. That is, the photolysis group has a chemical structural formula of

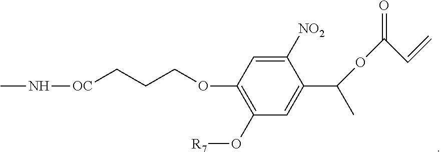

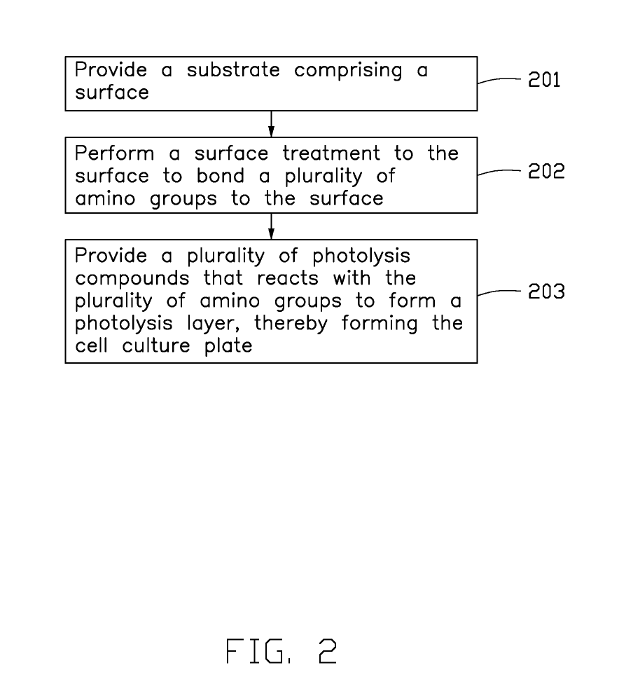

FIG. 2 illustrates a flowchart of a method for manufacturing a cell seeding plate 100 in accordance with an exemplary embodiment. The exemplary method is provided by way of example, as there are a variety of ways to carry out the method. Each block shown in FIG. 2 represents one or more processes, methods, or subroutines, carried out in the exemplary method. Furthermore, the illustrated order of blocks is by example only and the order of the blocks can change. Additional blocks may be added or fewer blocks may be utilized, without departing from this disclosure. The exemplary method can begin at block 201.

At block 201, a substrate 10 is provided. The substrate 10 is transparent, and made of plastic or glass, such as polystyrene.

At block 202, a surface 11 of the substrate 10 is performed by surface treatment, to cause a plurality of amino groups to be bonded to the surface 11.

In this exemplary embodiment, aminosilane compound (such as aminopropyl trimethoxysilane) are used in the surface treatment to bond the plurality of amino groups to the surface 11. In at least one exemplary embodiment, the plurality of amino groups can be bonded to the surface 11 by plasma synthesis.

At block 203, the plurality of amino groups are reacted with photolysis compounds to form a photolysis layer 13, thus forming the cell seeding plate 100. Each photolysis compound has a chemical structural formula of

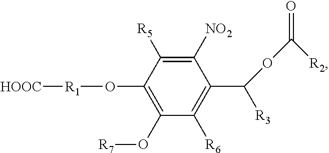

wherein R₁ and R₃ each represents alkane group, R₂ comprises alkane group or olefin group, R₅, R₆ and R₇ each represents hydrogen group or alkane group. The photolysis layer 13 comprises a plurality of photolysis groups. Each photolysis group has a chemical structural formula of

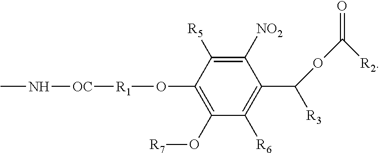

In at least one exemplary embodiment, R₂ of each photolysis compound can further be bonded to collagen, hyaluronic acid, protein for functionalizing cell, or peptides for functionalizing cell.

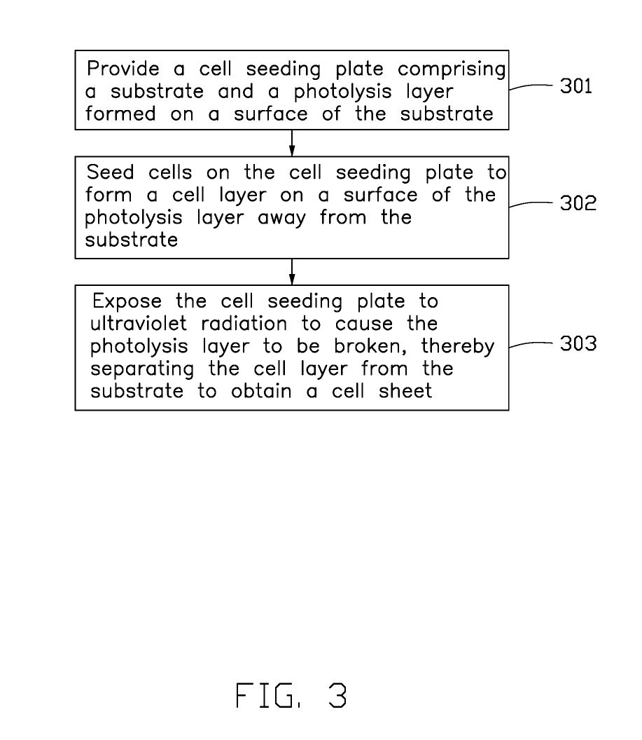

FIG. 3 illustrates a flowchart of a cell sheet separating method using the cell seeding plate 100. The exemplary method can begin at block 301.

At block 301, referring to FIG. 1, a cell seeding plate 100 is provided. The cell seeding plate 100 comprises a substrate 10 and a photolysis layer 13 formed on a surface 11 of the substrate 10.

The substrate 10 is transparent, and made of plastic or glass, such as polystyrene.

The photolysis layer 13 comprises a plurality of photolysis groups. Each photolysis group has a chemical structural formula of

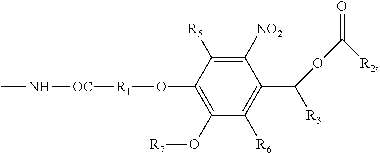

wherein R₁ and R₃ each represents alkane group, R₂ comprises alkane group or olefin group, R₅, R₆ and R₇ each represents hydrogen group or alkane group. Each photolysis group is bonded to the surface 11 by an amide group of the photolysis group.

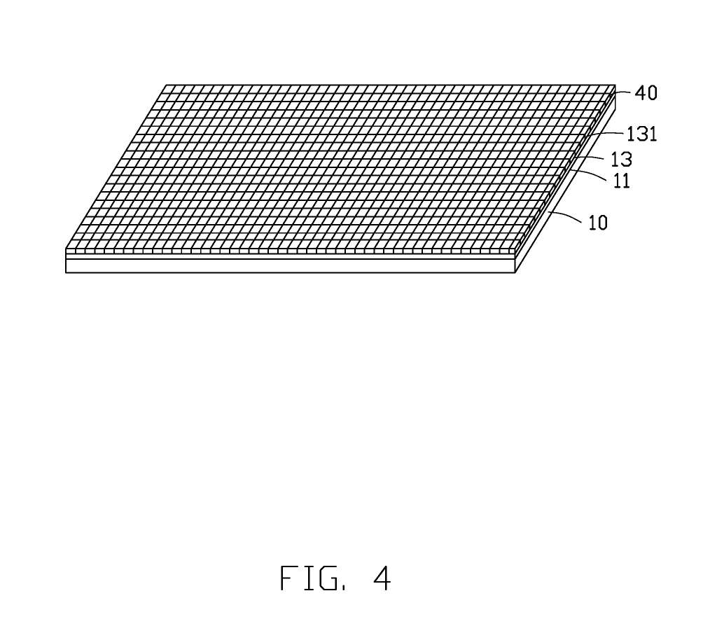

At block 302, cells are seeded on the cell seeding plate 100 to form a cell layer 40 (shown in FIG. 4) on a surface 131 of the photolysis layer 13 away from the substrate 10.

At block 303, the cell seeding plate 100 is exposed to ultraviolet radiation to cause the ester group of each photolysis group to be broken, so the cell layer 40 is separated from the substrate 10 to obtain a cell sheet.

Depending on the embodiment, certain of the steps of methods described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A cell seeding plate comprising:
a substrate comprising a surface; and
a photolysis layer formed on the surface;
wherein the photolysis layer comprises a plurality of photolysis groups, each of the plurality of photolysis groups has a chemical structural formula of 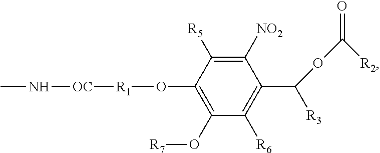
R₁ and R₃ each represents alkane group, R₂ comprises alkane group or olefin group, R₅, R₆ and R₇ each represents hydrogen group or alkane group, each of the plurality of photolysis groups is bonded to the surface by the amide group.

2. The cell seeding plate of claim 1, wherein the substrate is transparent, and made of plastic or glass.

3. The cell seeding plate of claim 1, wherein R₂ is a natural polymer comprising sulfur group or a synthetic polymer comprising sulfur group, the natural polymer is selected from collagen, hyaluronic acid, protein for functionalizing cell, or peptides for functionalizing cell.

4. The cell seeding plate of claim 1, wherein the photolysis group has a chemical structural formula of

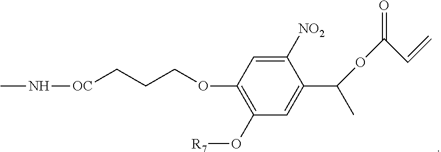

5. A method for manufacturing a cell seeding plate comprising:
providing a substrate comprising a surface;
performing a surface treatment to the surface to bond a plurality of amino groups to the surface; and
providing a plurality of photolysis compounds that reacts with the plurality of amino groups to form a photolysis layer, thereby forming the cell seeding plate, wherein each of the plurality of photolysis compounds has a chemical structural formula of 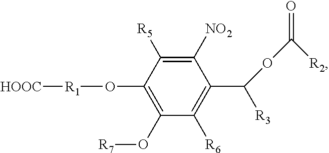
R₁ and R₃ each represents alkane group, R₂ comprises alkane group or olefin group, R₅, R₆ and R₇ each represents hydrogen group or alkane group, the photolysis layer comprises a plurality of photolysis groups, each of the plurality of photolysis groups has a chemical structural formula of

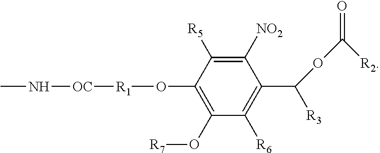

6. The method of claim 5, wherein the substrate is transparent, and made of plastic or glass.

7. The method of claim 5, wherein the photolysis group has a chemical structural formula of

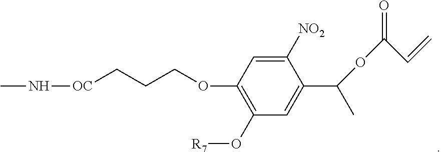

8. A cell sheet separating method comprising:
providing a cell seeding plate comprising:
a substrate comprising a surface; and
a photolysis layer formed on the surface;
wherein the photolysis layer comprises a plurality of photolysis groups, each of the plurality of photolysis groups has a chemical structural formula of 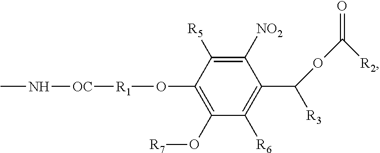
R₁ and R₃ each represents alkane group, R₂ comprises alkane group or olefin group, R₅, R₆ and R₇ each represents hydrogen group or alkane group, each of the plurality of photolysis groups is bonded to the surface by the amide group;
seeding cells on the cell seeding plate to form a cell layer on a surface of the photolysis layer away from the substrate; and
exposing the cell seeding plate to ultraviolet radiation to cause the ester group of the photolysis layer to be broken, thereby separating the cell layer from the substrate to obtain the cell sheet.

9. The cell sheet separating method of claim 8, wherein the substrate is transparent, and made of plastic or glass.

10. The cell sheet separating method of claim 8, wherein the photolysis group has a chemical structural formula of [chemical structure]
